(19) United States
(12) Patent Application Publication
(10) Pub. No.: US 2020/0060618 A1
(43) Pub. Date: Feb. 27, 2020

(54) ENCAPSULATED WEARABLE DEVICE AND SYSTEM WITH INCREASED SURFACE ENERGY

(71) Applicant: MC10, Inc., Lexington, MA (US)

(72) Inventors: Daniel Davis, Worcester, MA (US); David Garlock, Derry, NH (US); Steven Fastert, Chelmsford, MA (US)

(21) Appl. No.: 16/461,127

(22) PCT Filed: Nov. 14, 2017
(86) PCT No.: PCT/US2017/061474

§ 371 Date: May 15, 2019

Related U.S. Application Data
(60) Provisional application No. 62/422,340, filed on Nov. 15, 2016.

(51) Int. Cl. A61B 5/00

(52) U.S. Cl. CPC A61B 5/6833; A61B 5/6801

(57) ABSTRACT

Aspects of the present disclosure include a polymer matrix that is formed on a wearable device to increase the surface energy and reduce the tackiness of the surface of the wearable device. The present disclose includes a wearable device that can be worn on a user, such as one the user's skin. The device includes one or more electronic components and an encapsulation layer surrounding the one or more electronic components. The device further includes a polymer matrix at least partially covering a first side of the wearable device. The polymer matrix has a higher surface energy than the encapsulation layer so as to improve adhesion with an adhesive layer. The present disclosure also includes a wearable device system that further includes the adhesive layer.

### CROSS-REFERENCE TO RELATED APPLICATIONS

The present application claims priority to and the benefit of U.S. Provisional Application No. 62/422,340, filed Nov. 15, 2016, and entitled, “ENCAPSULATED WEARABLE DEVICE AND SYSTEM WITH INCREASED SURFACE ENERGY,” which is incorporated by reference herein in its entirety.

### FIELD OF THE INVENTION

The present disclosure relates to encapsulated devices. In particular, the present disclosure relates to improving the adhesion between an adhesive element and an encapsulated device, such as a wearable electronic device.

### BACKGROUND OF THE INVENTION

Electronic devices are becoming more prevalent as the cost to manufacture such devices decreases. Similarly, the size and profile of electronic devices are becoming smaller as advancements are made to reduce the size of the individual internal components. Such advancements have allowed electronic devices to expand into many different fields and applications. One such field is the medical field. The smaller profile of electronic devices, in addition to their wide-range of functionality, has increased their use within the medical and/or physiological fields and, in particular, within the field of wearable devices.

Despite the foregoing advancements, a need still exists to protect the electronic devices from external factors, including water, temperature, wear, contaminants, etc. Coating or encapsulating the electronic devices with a protective film, coating, and/or layer can achieve the desired protection of the internal components. However, such protective films, coatings, and/or layers include several drawbacks. For example, such films, coatings, and/or layers tend to be tacky and can increase the amount of debris (e.g., dirt, dust, dander, pollen, fungus, etc.) that sticks to or collects on the exteriors of the devices. In addition, such films, coatings, and/or layers can increase the coefficient of friction and/or tackiness with other materials (e.g., clothing). Such an increase in the coefficient of friction and/or tackiness can cause the device to stick to clothing and to be accidentally removed from a desired location. This can be particularly problematic for wearable devices. Such wearable devices can be worn on the surface of a user and can often come into contact with objects that cause the wearable device to separate from the user. The increase in the coefficient of friction and/or tackiness can cause the wearable device to be more likely separated from the user upon incidental contact.

Wearable devices can include a permanent adhesive layer that adheres the devices to surfaces, such as the skin of a user. Alternatively, wearable devices can be paired with removable adhesive elements, such as removable adhesive layers, patches, and/or stickers. The removable adhesives elements allow the same wearable device to be re-used by replacing the adhesive elements. However, the protective films, coatings, and/or layers of the wearable devices can prevent the replaceable adhesive elements from adequately adhering to the wearable device. Although the strength of the adhesive of the adhesive layer can be increased, this may create issues that prevent a clean removal of the replaceable adhesive element for re-use with the wearable device.

Therefore, there is a continuing need for developing materials and methods that solve the above and related problems.

### SUMMARY OF THE INVENTION

Aspects of the present disclosure include a polymer matrix that is formed on an exterior of an encapsulated device, such as a silicone encapsulated wearable device, to increase the surface energy of the encapsulated device and reduce tackiness.

Additional aspects of the present disclosure include a wearable device configured to be attached to a user to sense data regarding the user. The device includes one or more electronic components and an encapsulation layer surrounding the one or more electronic components and a polymer matrix partially covering a first side of the wearable device. The polymer matrix has a higher surface energy than the encapsulation layer, and the first side faces a surface upon which the wearable device is adhered.

Further aspects of the present disclosure include a wearable device system configured to be attached to a user to sense data regarding the user. The system includes a wearable device and an adhesive layer. The wearable device includes one or more electronic components and an encapsulation layer surrounding the one or more electronic components. The device further includes a polymer matrix at least partially covering a first side of the wearable device. The system further includes an adhesive layer configured to adhere to the first side of the wearable device. The first side is configured to face a surface (e.g., the skin of a user) upon which the wearable device is to be applied or adhered. The polymer matrix coating has a higher surface energy than the outer surface of the encapsulation layer and the adhesive layer adheres better to the polymer matrix coating than the outer surface of the encapsulation layer.

Additional aspects of the disclosure will be apparent to those of ordinary skill in the art in view of the detailed description of various embodiments, which is made with reference to the accompany drawings, a brief description of which is provided below.

### BRIEF DESCRIPTION OF THE DRAWINGS

The disclosure will be better understood from the following description of exemplary embodiments together with reference to the accompanying drawings.

FIG. 1 shows a wearable device having an array of discrete islands formed of a polymer matrix, in accord with aspects of the present disclosure.

FIG. 2 shows a wearable device having an alternative array of discrete islands formed of a polymer matrix, in accord with aspects of the present disclosure.

FIG. 3 shows a wearable device having an alternative array of discrete islands formed of a polymer matrix, in accord with aspects of the present disclosure.

FIG. 4 shows a wearable device having electrical contacts and an alternative array of discrete islands formed of a polymer matrix, in accord with aspects of the present disclosure.

FIG. 5A is a top view of a wearable device system 500, in accord with aspects of the present disclosure.

FIG. 5B is a cross-section view of the system of FIG. 5B through the line 5B-5B, in accord with aspects of the present disclosure.

### DETAILED DESCRIPTION OF THE ILLUSTRATED EMBODIMENTS

Although the present disclosure contains certain exemplary embodiments, it will be understood that the disclosure is not limited to those particular embodiments. On the contrary, the present disclosure is intended to cover all alternatives, modifications, and equivalent arrangements as may be included within the spirit and scope of the disclosure as further defined by the appended claims.

The present disclosure is directed to a polymer matrix (e.g., a coating) that can be applied to an encapsulated device to change the properties of the exterior surface of the encapsulation material. The polymer matrix coating can be used, for example, to make the encapsulation material less tacky or lower its coefficient of friction to aid in preventing the incidental or accidental removal of the device through contact with clothing and external objects or surfaces. The polymer matrix can further aid in adhering a removable adhesive element to the device, such as an adhesive layer, patch, sticker, and the like. The present disclosure is also directed to wearable devices including the polymer matrix, and a wearable device system that includes a wearable device having the polymer matrix and a removable adhesive element that is configured to adhere to the wearable device.

The wearable devices of the present disclosure can include any type of electronic device that can be worn by a user, such as on the user's skin, clothing, and/or an object associated with the user. The wearable device can be worn on and/or near the user to collect data and/or information regarding the user, such as one or more one or more physical, physiological, biological, chemical characteristics, parameters, and/or indicators regarding the user. The wearable devices can include one or more components that collect the data and/or information, such as one or more sensors; one or more components that power the other components, such as one or more power sources (e.g., batteries, solar cells, etc.); and one or more components that control the other components, such as one or more processors.

The wearable devices are encapsulated with a material to protect the components of the wearable device. Such an encapsulation material can be formed of a material that is bendable, flexible, and/or elastic (e.g., stretchable) or inelastic. Such an encapsulation material can be, for example, a silicone material. However, such additional encapsulation materials can be, for example, acrylics, epoxies, urethanes, rubbers, polyesters, and nylons. In some aspects, the entire wearable device can be encapsulated. For example, all of the one or more components can be encapsulated by the encapsulation material. Alternatively, one or more components, or one or more portions of the one or more components can be exposed relative to the encapsulation material. For example, the wearable device can include one or more electrodes that are configured to be in electrical contact with the user when the wearable device affixed to the user. In such as case, the electrodes can be exposed by the encapsulation material.

To solve the above-addressed issues, including, for example, reducing the tackiness and increasing the surface energy of the wearable devices, the wearable devices include a polymer matrix in or applied to their exteriors. In some aspects, at least a portion of the polymer matrix can be distinct from any polymer that forms the encapsulation material. In some aspects, at least a portion of the polymer matrix can be formed on the encapsulation material. In some aspects, at least a portion of the polymer matrix can be formed embedded or mixed within the encapsulation material but exposed to the exterior surface of the wearable device. For example, the exterior surface of the polymer matrix embedded within the encapsulation material can be co-planar with the exterior surface of the encapsulation material.

The polymer matrix applied to the wearable device results in the outer surface of the wearable device having a higher surface energy than the encapsulation material. The polymer matrix treated outer surface having a higher surface energy as compared to the encapsulation layer aids in adhering an adhesive element to the wearable device to adhere the wearable device to, for example, a user (e.g., the user's skin, clothing, etc.). The polymer matrix treated outer surface can also reduce the tackiness of the surface of the wearable device as compared to the encapsulation material. The reduced tackiness decreases the chances of incidental removal of the wearable device through accidental or elevated contact between the wearable device and an external object, such as the user's clothing, an object adjacent to the user, such as a piece of furniture or another individual, and the like.

The polymer matrix of the present disclosure can be formed of one or more poly(p-xylylene) polymers. In some aspects, the one or more poly(p-xylylene) polymers can be one or more parylene polymers, including, for example, parylene polymers according to the trade names Parylene C, Parylene D, Parylene F, and Parylene N. The one or more poly(p-xylylene) polymers are stable at normal operating conditions for wearable devices (e.g., ambient temperature, pressure, humidity, etc.), are substantially non-reactive, and are biocompatible. The polymer matrix formed of the poly(p-xylylene) polymers has a surface energy of about 19 mN/m to about 60 mN/m depending upon how the parylene is functionalized, with a minimum surface energy of about 35 mN/m representing the crossover point from low surface energy to higher surface energy materials. In contrast, the surface energy of the encapsulation material, such as silicone, has a surface energy of about 24 mN/m. Based on the difference in the surface energies, an adhesive element, such as a pressure sensitive acrylic-based adhesive layer, adheres better to the polymer matrix than the encapsulation material. The better adherence allows a system of the wearable device and the adhesive layer to perform better, such as in terms of better release of a second release layer with the adhesive element adhered to the wearable device, better adherence during use, and a cleaner release of the adhesive element when use of the element is finished. The better adherence of the polymer matrix in general allows different adhesive elements to be used with the wearable device, such as less expensive and/or less painful patches and/or stickers for a user to remove.

The polymer matrix can be applied over the entire wearable device (e.g., all outer surfaces), such as treating, coating or covering the entire outer surface of the encapsulation material. Alternatively, the polymer matrix can be applied such that it covers only a portion of the wearable device (e.g., by masking or selectively applying), such as covering only a portion of the encapsulation material or being embedded within the encapsulation material so as to cover only a portion of the exterior surface of the wearable device.

In some aspects, the wearable device can be considered to have at least two surfaces: (1) a skin side or bottom surface and (2) an air side or top surface. As described, the skin side or bottom surface is intended to face and adhere to the skin of the user, albeit not necessarily touching the skin based on the optional presence of an adhesive element between the skin and the wearable device. In some aspects, and as described above, the skin side or bottom surface can include one or more exposed components, such as one or more electrodes. Conversely, the air side or top surface of the wearable device is opposite from the bottom surface and is intended to face away from the user. When the wearable device is worn on the body, for example, under the clothing, the top surface can come in contact with the clothing and/or external objects that the body may come in contact with.

The top surface is where most of the incidental contact between the wearable device and an external object can occur. In aspects where the polymer matrix covers only a portion of the wearable device, the polymer matrix can cover the entire top surface. The polymer matrix covering the entire top surface lowers the tackiness of the wearable device relative to the encapsulation layer covering the top surface. The lower tackiness can reduce or prevent the chances of the wearable device from being removed from an object (e.g., a user) to which it is affixed through incidental contact. This can reduce the necessity of re-applying the device with a fresh skin adhesive element or requiring a significantly higher adhesion with higher pain upon removal from the skin.

Alternatively, in aspects where the polymer matrix covers only a portion of the wearable device, the polymer matrix can cover only a portion of the top surface. Such a portion can be about 99%, 95%, 90%, 80%, 75%, 66%, or 50% of the top surface. Such fractional coverage of the top surface can still reduce and/or prevent incidental contact from removing the wearable device, while not covering the entire top surface.

Where the polymer matrix covers only a portion of the wearable device, the polymer matrix can be formed on the wearable device so as to cover only a portion of the bottom surface. The portion of the bottom surface that is covered by the polymer matrix can be controlled so as to control the adhesive between the wearable device and an adhesive layer, patch, and/or sticker.

In some aspects, the polymer matrix can be irregularly or regularly distributed on the encapsulation layer, both on the top and bottom surfaces. The regular distribution of the polymer matrix can form a pattern. The pattern can be configured to assist in the adherence of an adhesive layer, patch, and/or sticker to the wearable device, such as on the bottom surface. In some aspects, the pattern can include one or more lines, such as parallel straight or curved lines, intersecting straight or curved lines, uniformly or non-uniformly spaced apart curved lines (e.g., spiral configurations), and the like. Such intersecting lines can be orthogonal lines or non-orthogonal lines. In some aspects, the pattern can be a plurality of discrete islands of the polymer matrix. Such islands can have various regular or irregular shapes. The regular shapes can include, for example, circles (dots), triangles, squares, rectangles, pentagons, hexagons, etc. The islands can all be the same shape or can have different shapes.

A pattern of regularly shaped discrete islands of the polymer matrix can form an array. The array can have one or more (e.g., 1, 2, 3, 4, 6, or more) columns of the discrete islands and one or more (e.g., 1, 2, 3, 4, 5, 6, or more) rows of the discrete islands. Each discrete island can be uniformly spaced/distributed apart or can be non-uniformly spaced/distributed apart.

As part of a wearable device system, an adhesive layer, patch, and/or sticker can be used to affix the wearable device to a user. The adhesive layer can be a single layer with adhesive on both sides, or can be a plurality of layers with adhesive layers on the top and bottom surfaces. For example, the middle layers can be moisture wicking layers, and the top and bottom adhesive layers can have apertures there through to wick away moisture.

The adhesive layer can be made of various adhesives. For at least the skin side, the adhesives can be bio-compatible and non-reactive adhesives. In some aspects, the adhesive layer can be formed of a pressure-sensitive adhesive. The pressure sensitive adhesive can be formed of elastomers based on acrylics, butyl rubber, ethylene-vinyl acetate (EVA), silicone, natural rubber, nitriles, and the like. In some aspects, the pressure-sensitive adhesives are preferably acrylic-based pressure-sensitive adhesives, including organic-based acrylate pressure-sensitive adhesives.

Although the application of the polymer matrix decreases the tackiness of the outer surface of the wearable device relative to the untreated outer surface of the encapsulation layer, the higher energy of the polymer matrix treated outer surface increases the adhesiveness of the adhesive layer to the outer surface of the wearable device. Acrylic-based adhesives do not possess initial wetting action on the encapsulation material, such as silicone. Accordingly, after a short time, the acrylic-based adhesives wet out to the encapsulation material sufficiently, but the initial application of the acrylic-based adhesive to the silicone surface and subsequent liner removal presents an issue. However, the application of polymer matrix to the wearable device, such as co-planar with or over the encapsulation material, greatly increases the surface energy of the surface allowing for very quick wetting of the acrylic-based adhesive layer to the polymer matrix. The quick wetting allows for improved adhesion of the adhesive layer and easy removal of the secondary release liner. Indeed, the surface energy can be so greatly increased by the polymer matrix that selective patterning of the polymer matrix can be used to control the amount of adhesion required to remove the secondary liner and allow the adhesive layer to be cleanly removed after the intended use.

The polymer matrix can be applied to the wearable device using vacuum deposition, such as vapor deposition polymerization (VDP). Using vacuum deposition, the polymer matrix is deposited as a vapor and surrounds the wearable device, penetrating and coating small cracks, crevices, and openings. The polymer matrix can be ultrathin and pinhole-free with a uniform thickness even on irregular surfaces. The deposition can occur at room temperature.

Through vacuum deposition, the exposed areas of the wearable device are coated with the polymer matrix. A masking can be used to apply the polymer matrix according to a specific pattern. For example, masking can cover areas of the wearable device where the polymer matrix is not desired and the wearable device can then be exposed to the polymer matrix through the vacuum deposition. After vacuum deposition, the masking can be removed to reveal the areas of the wearable device that are not covered by the polymer matrix. The resulting polymer matrix can be formed to be about 2.7 to about 3.3 microns thick, such as about 3 microns.

In some aspects, the VDP can begin with the polymer matrix in the form of a dimer is placed into a vaporizing chamber. The polymer matrix is heated, sublimating it directly to a vapor, and then heated again until the dimer cracks into a monomeric vapor. The vapor flows into an ambient-temperature deposition chamber kept at a medium vacuum (e.g., 0.1 Torr) where it spontaneously polymerizes onto all surfaces of the wearable device (and mask, if present), forming an ultrathin, uniform film. In some aspects, a cold trap (e.g., −90° to −120° C.) can be used to remove all residual polymer materials pulled through the coating chamber.

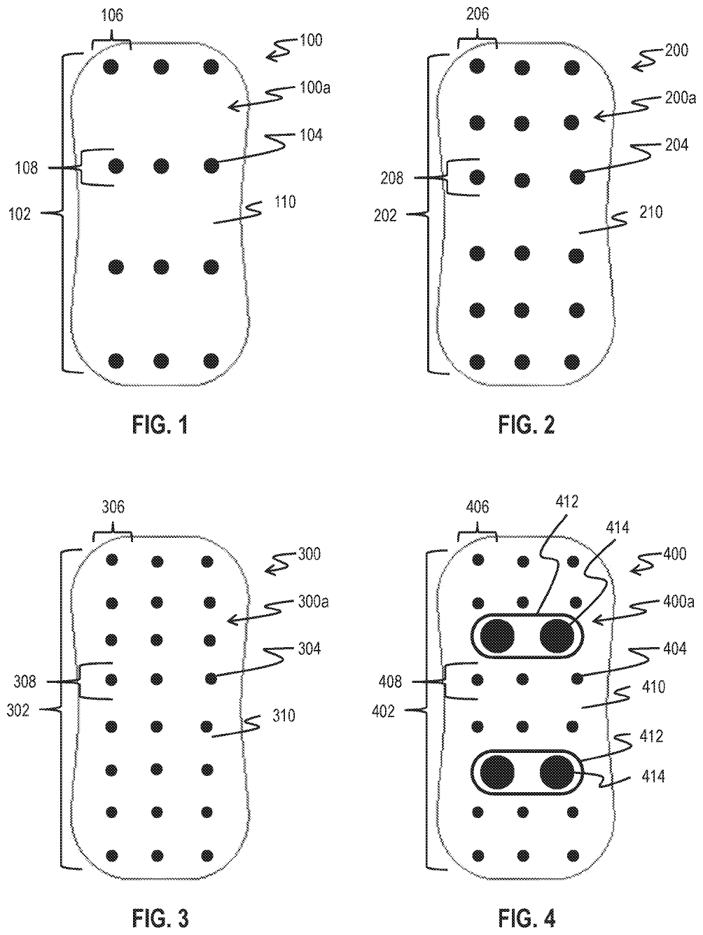

Based on the foregoing, FIGS. 1-4 show exemplary wearable devices according to aspects of the present disclosure. In particular, FIG. 1 shows a surface 100a of a wearable device 100 having an array of dots 104 formed of a polymer matrix as described above, in accord with aspects of the present disclosure. More specifically, the wearable device 100 includes an array 102 of 12 dots 104 on the surface 100a. The array 102 includes three columns 106 and four rows 108. The surface 100a of the device 100 also includes the exposed encapsulation layer 110 surrounding the dots 104. As described above, the encapsulation layer 110 can be formed of, for example, silicone or a similar material. The exposed silicone layer 110 can either surround and be co-planar with the dots 104 or can entirely encapsulate the wearable device 100 and be under the dots 104.

The size of the dots 104 can vary depending on the desired surface area of the dots 104 (and the desired total surface energy) relative to the total surface area of the surface 100a, or relative to the surface area of the exposed encapsulation layer 110. In some aspects, the diameters of the dots 104 can be about 0.25 mm to about 5 mm, such as about 1 mm, or about 2 mm, or about 3 mm. The diameters of the dots 104 can all be substantially the same diameter or can be different diameters. For example, some of the dots 104 can have a diameter of about 1 mm and some of the dots 104 can have a diameter of about 3 mm. In some aspects, the different diameters of the dots 104 can vary depending on the locations of the dots 104 relative to the device 100. For example, the dots 104 near the perimeter of the device 100 can have a smaller or a larger diameter than the dots 104 near the center of the device 100.

As mentioned above, the diameters of the dots 104 can be selected to control the combined surface area of the dots 104 relative to the total surface area of the surface 100a of the device 100 or, correspondingly, the surface area of the exposed encapsulation layer 110. In some aspects, the surface area of the dots 104 can be about 0.25% to about 10% of the total surface area of the surface 100a of the device 100. At this percent of coverage, the dots 104 allow for an adhesive layer, patch, and/or sticker to adhere to the surface 100a while still being cleanly removed from the wearable device 100 for re-use of the wearable device 100 with another adhesive layer and/or path. Where the dots 104 have a diameter of about 1 mm, the total surface area of the dots 104 can be about 0.46% of the total surface area of the surface 100a of the device 100. Where the dots 104 have a diameter of about 2 mm, the total surface area of the dots 104 can be about 1.84% of the total surface area of the surface 100a of the device 100. Where the dots 104 have a diameter of about 3 mm, the total surface area of the dots 104 can be about 4.14% of the total surface area of the surface 100a of the device 100.

In addition to adjusting the size of the dots 104 to control the ratio of the combined surface area of the dots 104 relative to the total surface area of the surface 100a, the size of the array 102 can also be adjusted.

FIG. 2 shows a wearable device 200 having an alternative array 202 of dots 204 formed of a polymer matrix, in accord with aspects of the present disclosure. The array 202 includes 18 dots 204 on the surface 200a of the device 200. The array 202 includes three columns 206 and six rows 208. The surface 200a of the device 200 also includes the exposed encapsulation layer 210 surrounding the dots 204.

The diameters of the dots 204 can be the same diameters as the dots 104 discussed above. Thus, based on the larger number of dots 204 (e.g., 18 rather than 12), the percentage of the total surface area of the surface 200a covered by the dots 204 can be larger. For example, where the dots 204 have a diameter of about 1 mm, the total surface area of the dots 204 can be about 0.69% of the total surface area of the surface 200a of the device 200. Where the dots 204 have a diameter of about 2 mm, the total surface area of the dots 204 can be about 2.76% of the total surface area of the surface 200a of the device 200. Where the dots 204 have a diameter of about 3 mm, the total surface area of the dots 204 can be about 6.22% of the total surface area of the surface 200a of the device 200.

FIG. 3 shows a wearable device 300 having an alternative array 302 of dots 304 formed of a polymer matrix, in accord with aspects of the present disclosure. The array 302 includes 24 dots 304 on the surface 300a of the device 300. The array 302 includes three columns 306 and six rows 308. The surface 300a of the device 300 also includes the exposed encapsulation layer 310 surrounding the dots 304.

The diameters of the dots 304 can be the same diameters as the dots 104 and 204 discussed above. Thus, based on the larger number of dots 304 (e.g., 24 rather than 12 or 18), the percentage of the total surface area of the surface 300a covered by the dots 304 can be larger. For example, where the dots 304 have a diameter of about 1 mm, the total surface area of the dots 304 can be about 0.92% of the total surface area of the surface 300a of the device 300. Where the dots 304 have a diameter of about 2 mm, the total surface area of the dots 304 can be about 3.14% of the total surface area of the surface 300a of the device 300. Where the dots 304 have a diameter of about 3 mm, the total surface area of the dots 304 can be about 8.3% of the total surface area of the surface 300a of the device 300.

FIG. 4 shows a wearable device 400 having an array 402 of dots 404 formed of a polymer matrix, in accord with aspects of the present disclosure. Similar to FIG. 2, the array 402 includes 18 dots 404 in three columns 406 and six rows 408. The dots 404 are formed on the surface 400a of the device 400. Also similar to above, the surface 400a of the device 400 includes an exposed encapsulation layer 410.

The surface 400a of the device 400 also includes apertures 412 through the encapsulation layer 410. In some aspects, the device 400 can include two apertures 412, as shown. However, the device 400 can include one or more than two apertures 412, such as three, four, five, six, seven or more apertures 412. The apertures 412 expose electrical contacts 414. In some aspects, each of the apertures 412 can expose two electrical contacts 414, as shown. However, the device 400 can include one or more than two electrical contacts 414 for each of the apertures 412. In some aspects, each of the apertures 412 can include different numbers of electrical contacts 414.

With the presence of the apertures 412, the number and the size of the dots 404 can be the same as the number and the size of the dots without the apertures. For example, the number and size of the dots 404 can be the same as the number and size of the dots 204 in FIG. 2. The difference between the devices 200 and 400 can merely be the arrangement of the dots 404 on the surface 400a of the device 400, such as the arrangement accounting for the apertures 412. Alternatively, the number and/or the size of the dots 404 can be modified to account for the loss of the surface area of the encapsulation layer 410 or, more particularly, the loss of the available surface area for an adhesive to attach to the surface 400a of the device 400. For example, the number of dots 404 can be increased, or the size of the dots 404 can be increased, or both, to account for the loss of surface area of the encapsulation layer 410.

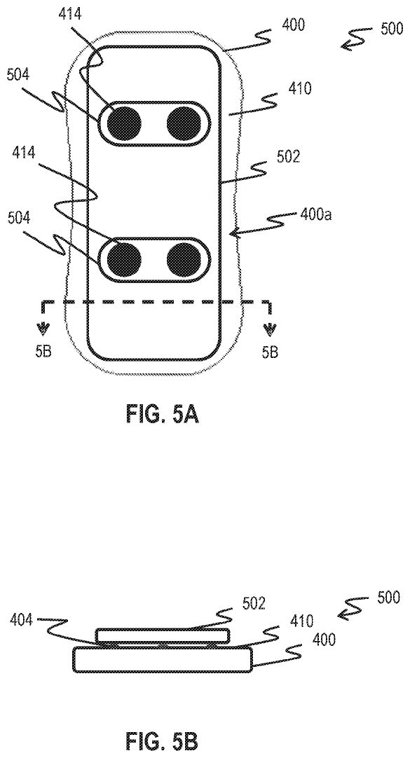

FIGS. 5A and 5B show the wearable device 400 of FIG. 4 as part of a wearable device system 500, in accord with aspects of the present concepts. Specifically, FIG. 5A is a top view of the wearable device system 500, and FIG. 5B is a cross-section view of the system 500 through the line 5B-5B in FIG. 5A. The wearable device system 500 includes the wearable device 400 of FIG. 4 with the surface 400a and the exposed encapsulation layer 410. Adhered to the surface 400a of the wearable device 400 is an adhesive layer 502, as described above. Because the wearable device 400 includes the exposed electrical contacts 414, the adhesive layer 502 includes apertures 504 to expose the electrical contacts 414. However, in aspects in which the wearable device does not include exposed components, such as exposed electrical contacts, the adhesive layer can omit the apertures 504.

Based on the presence of the dots 404 (FIG. 4), the adhesive layer 502 can adhere to the surface 400a better than if the surface 400a was merely the encapsulation layer 410. The dots 404 can be formed on the encapsulation layer 410, as shown in FIG. 5B. Alternatively, as described above, the dots 404 can be formed within and co-planar with the encapsulation material.

Based on the foregoing, the polymer matrix of the present disclosure assists in preventing the incidental or accidental removal of the device by removing the tackiness of the exposed surface as compared to the encapsulation material. The polymer matrix further aids in adhering a removable adhesive layer, patch, and/or sticker to the device. In some aspects, the polymer matric can also protect one or more exposed components, or components under the polymer layer, such as, for example, pad printed graphics on the encapsulation material surface. The polymer matrix can reduce or prevent the pad printed graphics from wearing off. The polymer matrix can also provide an additional barrier to external factors, such as liquids. The reduced tackiness als0 makes the wearable device more easily cleaned, such as with just soap and water, which can maintain a quality aesthetic appearance.

Other embodiments are within the scope and spirit of the present disclosure. Further, while the description above refers to the invention, the description may include more than one invention.

1. A wearable device configured to be attached to a user to sense data regarding the user, the device comprising:
one or more electronic components;
an encapsulation layer surrounding the one or more electronic components; and
a polymer matrix at least partially covering a first side of the wearable device,
wherein the polymer matrix has a higher surface energy than the encapsulation layer, and the first side faces a surface upon which the wearable device is adhered.

2. The wearable device of claim 1, wherein the encapsulation is formed of silicone.

3. (canceled)

4. The wearable device of claim 1, wherein the polymer matrix forms one or more discrete islands on the first side of the wearable device.

5. The wearable device of claim 4, wherein the one or more discrete islands are dots.

6. The wearable device of claim 4, wherein the one or more discrete islands are formed on the encapsulation layer, embedded or mixed within and co-planar with the encapsulation layer, or a combination thereof.

7. (canceled)

8. The wearable device of claim 4, wherein the one or more discrete islands are 1 to 5 microns thick.

9. The wearable device of claim 4, wherein the one or more discrete islands form an array of device islands.

10. The wearable device of claim 9, wherein the array is a three column by at least four row array of the dots distributed evenly across the first surface.

11-13. (canceled)

14. The wearable device of claim 13, wherein the dots cover at least about 0.46% of the surface area of the first surface.

15. (canceled)

16. (canceled)

17. The wearable device of claim 5, wherein the dots are about 2 millimeter in diameter.

18. The wearable device of claim 17, wherein the dots cover at least about 1.84% of the surface area of the first surface.

19. (canceled)

20. (canceled)

21. The wearable device of claim 5, wherein the dots are about 3 millimeter in diameter.

22. The wearable device of claim 21, wherein the dots cover at least about 4.14% of the surface area of the first surface.

23. (canceled)

24. (canceled)

25. The wearable device of claim 1, wherein the polymer matrix entirely covers a second surface of the encapsulation layer, wherein the second surface is opposite from the first surface.

26. A wearable device system configured to be attached to a user to sense data regarding the user, the system comprising:
a wearable device comprising:
one or more electronic components;
an encapsulation layer surrounding the one or more electronic components; and
a polymer matrix at least partially covering a first side of the wearable device; and
an adhesive layer configured to adhere to the first side of the wearable device,
wherein the polymer matrix has a higher surface energy than the encapsulation layer, the adhesive layer adheres better to the polymer matrix than the encapsulation layer, and the first side faces a surface upon which the wearable device is adhered.

27. (canceled)

28. The system of claim 26, wherein the polymer matrix forms a pattern on the first side of the wearable device.

29. The system of claim 28, wherein the pattern is a plurality of lines, an array of discrete dots, or a combination thereof.

30. (canceled)

31. (canceled)

32. The system of claim 31, wherein the array of dots comprises three columns and at least four rows.

33. (canceled)

34. (canceled)

35. The system of claim 26, wherein the polymer matrix is formed on the encapsulation layer on the first side of the wearable device.

36. The system of claim 35, wherein the polymer matrix covers substantially all of the encapsulation layer on a second side of the wearable device opposite from the first side.

37-39. (canceled)
